(19) United States
(12) Patent Application Publication
(10) Pub. No.: US 2022/0282001 A1
(43) Pub. Date: Sep. 8, 2022

(54) STABILIZATION OF PROTEINS WITH CYCLODEXTRINS

(71) Applicant: CYCLOLAB CYCLODEXTRIN RESEARCH AND DEVELOPMENT LABORATORY LTD., Budapest (HU)

(72) Inventors: Lajos Szente, Budapest (HU); Tamas Sohajda, Halasztelek (HU); István Puskás, Budapest (HU)

(73) Assignee: CYCLOLAB CYCLODEXTRIN RESEARCH AND DEVELOPMENT LABORATORY LTD., Budapest (HU)

(21) Appl. No.: 17/193,247

(22) Filed: Mar. 5, 2021

(51) Int. Cl. C08B 37/16; C07C 211/13

(52) U.S. Cl. CPC C08B 37/0012; C07C 211/13

(57) ABSTRACT

Present invention relates to the protection of peptides and proteins against aggregation.

The process and compositions according to the invention improve protein stability in presence of cyclodextrins without establishing any covalent interactions, only by inclusion and/or electrostatic phenomena. The carbohydrate compositions provide a temporary local molecular coating on the surface of protein macromolecules, via physical glycosylation.

### FIELD OF THE INVENTION

Present invention relates to the protection of peptides and proteins against physical aggregation.

The process and compositions according to the invention improve protein stability in presence of cyclodextrins without establishing any covalent interactions, only by inclusion and/or electrostatic phenomena. The carbohydrate compositions provide a temporary local molecular coating on the surface of protein macromolecules, via physical glycosylation.

### BACKGROUND OF THE INVENTION

The efficient stabilization of proteins has long been a challenge for pharmaceutical technologists. Among the number of technical possibilities, the utility of stabilizing excipients, primarily of non-ionic surfactants, such as Tween (Polyoxyethylene sorbitan monooleate), carbohydrate additives such as low molecular weight monosaccharides, sugar polymers, dextrans, cyclodextrins (CDs), heparinoids are the most frequently described additives in the literature.

In European patent EP 0787 497 Kobayashi et al. describes the use of water-soluble heterocyclic compounds such as creatinine, acetyl-tryptophane salts and nicotinamide for stabilizing human growth hormone.

In document US 2002/0146409 Herring et al. discloses stabilization of lyophilized blood proteins by hydroxypropylated alpha-cyclodextrin (HPACD).

In Chinese patent CN106199007, Hong et al. uses adenosine monophosphate, PVP 401, heparin sodium etc. for the stabilization of heat shock protein Hsp90 alpha protein.

PCT patent application WO 91/11200 by Konings et al. discloses the improvement of erythropoietin stability in formulations by using alpha-, beta-, gamma CDs and their hydroxyalkyl derivatives.

PCT patent application WO 90/03784 by Hora et al. relates to the cyclodextrin peptide complexes and methods for solubilization and stabilization of polypeptides.

Lee and Lin, published on the improvement of salmon calcitonin lyophilized solid form by using excipients trehalose, mannitol, larger polyols, dextrans, human serum albumin and hydroxypropyl-beta-cyclodextrin (HPBCD) (Process Biochemistry 46. 2011. 2163-2169.).

Utility of cyclodextrins in extraction and purification of proteins in particular membrane proteins has long been known and applied in laboratory practice. It this case the cyclodextrins are employed to selectively remove detergents applied during protein extractions.(Althof, T.; Davies, K. M.; Schulze, S.; Joos, F.; Kühlbrandt, W. Angew. Chem. 2012, 51(33), 8343-8347.)

Similar use has been described: the selective detergent-extraction from mixed detergent/lipid/protein micelles, using cyclodextrin inclusion compounds as a novel generic approach for the preparation of proteoliposomes. (Degrip, W. J.; Vanoostrum, J.; Bovee-Geurts, P. H. M. 1998. Biochem. J. 330(2), 667-674.)

Methyl-beta-cyclodextrin was used for controlled 2-dimensional crystallization of membrane proteins, here the presence of cyclodextrin improved highly ordered self-assembly process of monomeric species. (G. A. Signorell et al. J. Struct. Biol. 2007. 157(2), 321-328. Textor, M.; Keller, S. (2016) Chapter Six—Calorimetric Quantification of Cyclodextrin-Mediated Detergent Extraction for Membrane-Protein Reconstitution. Methods in Enzymology. 567, 129-156.)

Serno et al investigated the agitation-induced aggregation of immunoglobulins by using hydroxypropyl-beta-CD (HPBCD). In contrast to other studies with HBCD, protein stabilization could not be attributed to direct interaction between hydrophobic amino acids on the IgG and this excipient. Competition with the protein for the air-water interface appears to be the dominating mechanism of stabilization. (T. Serno, J. F Carpenter, T. W Randolph, G. Winter, J Pharm Sci. 2010 March; 99(3):1193-1206.)

Serno et al reviewed the beneficial use of cyclodextrins and their derivatives in protein formulations. Stabilization potentials of CDs are discussed in liquid as well as in dried formulations. The mechanisms of stabilization against different kinds of stress conditions, such as thermal or surface-induced, are discussed in detail. (T. Serno R. Geidobler G. Winter Adv Drug Deliv Rev. 2011 October; 63 (13):1086-1106.)

Protein self-assembly and aggregation in solutions have been known to affect negatively both their practical application and the physiological-pharmacological functions of these macromolecules.

The stabilization of biologicals such as therapeutic peptides, proteins monoclonal antibodies etc. is required to make these therapeutic agents druggable formulations with acceptable storage shelf-life and maintained therapeutic efficacy.

Moreover, certain protein aggregations are thought to be responsible for a number of important pathological conditions such as ageing, dementia, Alzheimer's disease etc.

### DETAILED DESCRIPTION

It is an object of the present invention to provide binary cyclodextrin combinations both as physical mixtures and inclusion complexes that show improved protein stabilizing effect. In an attempt to provide efficient stabilization for proteins, both in physical and chemical sense, it was found that the parenterally applicable cyclodextrins, such as hydroxypropylated-alpha-beta- and gamma-cyclodextrins showed some stabilizing effect, as expected and described in the prior art. The probable mechanism of this effect is based on the complex formation between lipophilic domains of the proteins (inclusion of the aromatic amino acid residues) and cyclodextrins. However, it was surprisingly found that if natural lipophiles that are known to form stable inclusion complexes with cyclodextrins, were combined with CDs, the protein stabilizing potency of these cyclodextrin-lipid complexes was higher than that achieved by the empty cyclodextrins. This positive effect was not expected, since in case of these lipid-cyclodextrin complexes, the cavities of cyclodextrins were already occupied by respective lipophiles. It is thought that the complexation-dissociation equilibrium in aqueous solutions was responsible for the enhanced protein stabilization provided by lipid-CD complexes.

Similar enhancement of the prevention of protein aggregation was found when ionic cyclodextrin derivatives (such as carboxyalkyl-, sulfoalkyl-, quaternary amino- etc. CDs), were combined with an oppositely charged, water-soluble small ionic compound, (e.g. nicotinamide, L-carnitine, spermine, creatinine, nucleosides and their phosphate esters).

This latter phenomenon is assumed to be due to the hydrotropic nature of the electrostatically combined cyclodextrin-counter ion binary systems in aqueous solution. Again, the protein stabilizing efficacy of the binary, charged system surpassed that achieved by ionic CDs and oppositely charged small molecules, alone.

Combination of Cyclodextrins With Lipophiles

The preferred embodiment of the invention: The host-guest type inclusion complexes of natural lipophiles (mainly fatty acids, sterols, carotenoids) formed with highly water-soluble cyclodextrins appear instantly water-soluble, amorphous solid formulations. The application of these lipid-cyclodextrin complexes was found to result in a protein stabilization against aggregation and precipitation. The below listed lipophiles were found as preferred, suitable artificial chaperons to improve protein stability in solution and in the solid state:

| | |
|---|---|
| cholesterol/beta-cyclodextrin methyl ether with a lipid content of | 3-5% |
| beta-sitosterol/beta-cyclodextrin methyl ether with a lipid content | 4.0% |
| of | |
| desmosterol/beta-cyclodextrin methyl ether with a lipid content of | 5.2% |
| lanosterol/beta-cyclodextrin methyl ether with a lipid content of | 4.4% |
| Na-palmitate/beta-cyclodextrin methyl ether with a lipid content of | 2.9% |
| ascorbyl-palmitate/beta-cyclodextrin methyl ether with a lipid | 2.0% |
| content of | |
| linolenic acid/beta-cyclodextrin methyl ether with a lipid content of | 2.1% |
| oleic acid/beta-cyclodextrin methyl ether with a lipid content of | 2.5% |
| lipoic acid/hydroxypropyl-beta-cyclodextrin with a lipid content of | 7.3% |

Another preferred embodiment of the invention is the combination of water-soluble anionic cyclodextrin derivatives with natural and synthetic nucleoside phosphates and oppositely charged small molecular entities, such as spermine, spermidine, L-carnitine, taurine etc. in their free base form.

### Combinations of Anionic Cyclodextrins With Nucleosides, Cationic Small Molecules

A binary combination of carboxyalkylated, sulfated, phosphated and sulfoalkylated cyclodextrins with nucleoside phosphates was found to provide a significantly improved stabilization effect of these combinations against protein deposit formation and aggregation in an aqueous system.

The preferred combinations according to the present invention are as follows:
carboxyalkyl (C1-C3) alpha, beta and gamma cyclodextrins
sulfoalkyl (C3-C4) ether alpha, beta and gamma cyclodextrins
succinyl-methyl-alpha, beta and gamma cyclodextrins
sulfated alpha, beta and gamma cyclodextrins
phosphated alpha, beta- and gamma cyclodextrins

The preferred combinations according to the present invention are as follows:
carboxyalkyl (C1-C3) alpha, beta, and gamma cyclodextrins
sulfoalkyl-(C3-C4) ether alpha, beta, and gamma cyclodextrins
and spermidine, spermine, L-carnitine, creatine and taurine

Patel et al. described ATP (adenosine triphosphate) in addition to being an energy source for biological reactions, for which micromolar concentrations are sufficient, that millimolar concentrations of ATP may act to keep proteins soluble (Science 356, 753-756, 2017). Nevertheless, this document does not provide teaching that the effect of ATP in combination with cyclodextrins may be superior to that observed in the presence of ATP itself.

The preferred ratios of the above combinations are in the range of 1:1 and 1:0.01 mole/mole between cyclodextrins and guest molecules as well as 1:1 and 1:0.01 mole/mole between small molecule cationic entities and cyclodextrins.

The physical glycosylation of proteins by using the above combinations both the water soluble lipid-cyclodextrin complexes and small cationic molecules combinations, were found to provide a partial non-covalent molecular coating of the less polar domains of proteins in water. This leads to the hindered, decelerated self-assembly process taking place between monomeric protein units.

Present invention directs to use of a cyclodextrin-lipid complex for enhancing the physical stability of a protein in aqueous environment characterized in that the molar ratio of the lipid to cyclodextrin is ranging from 0.01:1 to 1:1, particularly that the cyclodextrin-lipid complex comprises cyclodextrin selected from the family of unmodified, alkylated, hydroxyl-alkylated, carboxyalkylated, sulfoalkylated alpha- beta- and gamma-cyclodextrins and/or the the applied lipid is selected from sterols, steroids, phospholipids, fatty acids and their naturally occurring derivatives.

Present invention also directs to use of a complex constituting of a cyclodextrin and a nucleoside phosphate for enhancing the physical stability of a protein in aqueous environment characterized in that the molar ratio of the nucleoside phosphate to cyclodextrin is ranging from 0.01:1 to 1:1, particularly that the a complex is constituting of a cyclodextrin selected from the family of unmodified, alkylated, hydroxyl-alkylated, carboxyalkylated, sulfoalkylated alpha-beta- and gamma-cyclodextrins and/or said nucleoside is selected from natural- and synthetic nucleoside analogue phosphates of different number of phosphate groups, particularly adenosine triphosphate or adenosine diphosphate.

Present invention further directs to use cyclodextrin-amine complex for enhancing the physical stability of a protein in aqueous environment characterized in that the molar ratio of the amine to cyclodextrin is ranging from 0.01:1 to 1:1, particularly that the applied cyclodextrin is from the family of unmodified, alkylated, hydroxyl-alkylated, carboxyalkylated, sulfoalkylated alpha- beta- and gamma-cyclodextrins and/or the amine is selected from spermidine, spermine, L-carnitine, creatine and taurine.

The following examples are detailed for the illustration of the present invention without any limitation thereof.

### EXAMPLE 1

### Preparation and Use of Cholesterol/Methyl-Beta-Cyclodextrin (MBCD) Formulation For the Prevention of Protein Aggregation

5 grams of cholesterol and 96 grams of statistically methylated beta-cyclodextrin (MBCD) with an average methylation degree of 1.8 methyl groups per glucopyranose units were dissolved in 75 mL 96 V % ethanol at room temperature by ultrasonication. The common solution was evaporated to dryness in vacuo at 45° C., resulting in a white amorphous solid. This is then dissolved in 25 mL of purified water via ultrasonication, leading to a concentrated clear colorless solution. The water-solution is chilled to minus 60° C. and the ice-water is removed by freeze drying.

The resulting cholesterol-methyl-beta cyclodextrin complex appeared as a white amorphous powder. The cholesterol load of the complex was 4.6-4.7%, by HPLC, and the residual water content was 2.0% by weight (Karl-Fischer method). The cholesterol cyclodextrin complex was reconstituted in water by dissolving 1 gram in 5 mL of water. From this stock solution 3-20 mM amounts were added to lysozyme solutions and the stabilizing effect of the additive was tested at 60° C. heat treated lysozyme (heat treatment). The results of the study are listed in Table 1.

TABLE 1
Aggregation of lysozyme solution (determined by optical density
measurements at 405 nm) in the presence of cholesterol/MBCD.

| heat | Lysozyme aggregation (Absorbance at 405 nm) | | | | | | |
|---|---|---|---|---|---|---|---|
| treatment | control | | | 3 mM | 5 mM | 10 mM | 20 mM |
| at 60° C. | (no | 3 mM | MBCD | cholesterol | cholesterol | cholesterol | cholesterol in |
| (Hours) | additive) | cholesterol | only | in complex | in complex | in complex | complex |
| 0 | 0.020 | highly | 0.020 | 0.017 | 0.020 | 0.022 | 0.021 |
| 0.5 | 0.51 | turbid | 0.32 | 0.026 | 0.024 | 0.024 | 0.022 |
| 1.0 | 0.90 | suspension | 0.70 | 0.031 | 0.033 | 0.026 | 0.027 |
| 1.5 | 1.12 | | 0.84 | 0.054 | 0.050 | 0.044 | 0.047 |
| 2.0 | 1.78 | | 1.00 | 0.440 | 0.381 | 0.334 | 0.290 |

### EXAMPLE 2

### Beta-Sitosterol Methyl-Beta-Cyclodextrin Formulation Preparation and Use For Stabilization of Ovalbumin

4.5 grams of beta-sitosterol and 96 grams of methyl-beta-cyclodextrin were dissolved in 80 mL 96 V % ethanol upon intense agitation at room temperature. The resulting clear solution was further processed according to the steps described in Example 1. The lyophilized beta sitosterol methyl-beta-cyclodextrin formulation had a sterol content of 4.3%, and a residual water content of 3.5% (by Karl-Fischer titration)

1 gram of the complex formulation was re-dissolved in 5 mL of water and added in 15-45 mM amounts to the ovalbumin solution. The heat-induced aggregation of ovalbumin in presence of beta-sistosterol/methyl-beta-cyclodextrin formulation was followed by optical density measurements during a 60° C. heat treatment of the protein solutions. The results of the heat treatment are shown in Table 2.

TABLE 2
Aggregation of egg ovalbumin solutions in water during a heat treatment at 60° C. in the
presence of beta-sitosterol/methyl-beta-cyclodextrin formulation (by optical density at 405 nm)

| | Ovalbumin aggregation (absorbance at 405 nm) | | | | | | |
|---|---|---|---|---|---|---|---|
| heat | | | | 15 mM | 20 mM | | |
| treatment | control | 15 mM | | beta | beta | 30 mM beta | 45 mM beta |
| at 60° C. | (no | beta | MBCD | sitosterol in | sitosterol in | sitosterol in | sitosterol in |
| (Hours) | additive) | sitosterol | only | complex | complex | complex | complex |
| 0 | 0.018 | highly | 0.020 | 0.020 | 0.020 | 0.022 | 0.021 |
| 0.5 | 0.44 | turbid | 0.24 | 0.066 | 0.054 | 0.044 | 0.041 |
| 1.0 | 0.86 | suspension | 0.72 | 0.081 | 0.070 | 0.055 | 0.034 |
| 1.5 | 1.16 | | 0.84 | 0.120 | 0.077 | 0.067 | 0.055 |
| 2.0 | 1.87 | | 1.00 | 0.144 | 0.138 | 0.097 | 0.088 |

### EXAMPLE 3

### Preparation and Use of Desmosterol/Methyl-Beta-Cyclodextrin Formulation to Stabilize Protein Solutions

0.5 gram desmosterol (cholesta-5,24-diene-3β-ol) and 9.5 grams of methyl-beta-cyclodextrin (MBCD) were dissolved in 82 mL 96 V % ethanol with ultrasonication at room temperature.

Further processing was the same as described in detail in Example 1 of the present invention. The resulting desmosterol-MBCD complex had a desmosterol content of 4.2%, and the residual water content of 4.0% by weight.

The reconstituted solution of the sterol complex in concentrations of 10-25 mM added to the egg ovalbumin solutions was assayed for heat-induced aggregation.

The results are listed in Table 3.

TABLE 3
Aggregation of egg ovalbumin solutions in water during a heat treatment at 60° C. in
the presence of desmosterol/methyl-beta-cyclodextrin formulation (by optical density at 405 nm)

| heat | Ovalbumin aggregation (absorbance at 405 nm) | | | | | | |
|---|---|---|---|---|---|---|---|
| treatment | control | 10 mM | | 10 mM | 15 mM | 20 mM | 25 mM |
| at 60° C. | (no | desmo- | MBCD | desmosterol | desmosterol | desmosterol | desmosterol |
| (Hours) | additive) | sterol | only | in complex | in complex | in complex | in complex |
| 0 | 0.014 | highly | 0.022 | 0.020 | 0.020 | 0.022 | 0.021 |
| 0.5 | 0.46 | turbid | 0.28 | 0.050 | 0.044 | 0.036 | 0.028 |
| 1.0 | 0.77 | suspension | 0.74 | 0.180 | 0.093 | 0.088 | 0.074 |
| 1.5 | 1.11 | | 0.89 | 0.270 | 0.110 | 0.096 | 0.077 |
| 2.0 | 1.88 | | 1.02 | 0.320 | 0.166 | 0.088 | 0.088 |

### EXAMPLE 4

### Comparison of the Stabilizing Potency of Methyl-Beta-Cyclodextrin and Cholesterol-Methyl-Beta-Cyclodextrin Complex on the Aqueous Egg Ovalbumin Solutions Against Heat Treatment

Three test solutions were made and tested:

Solution I.: Egg ovalbumin (Sigma Aldrich) 35 mg was dissolved in 7 mL of simulated intestinal fluid (a phosphate buffer according to Ph.Eur. pH 7.4) at room temperature. This solution served as control test solution in the experiment.

Solution II.: The same composition of solution as above was made except that this contained also 350 mg of methyl-beta-cyclodextrin in the buffer.

Solution III.: A third composition was made in the same buffer system, from 35 mg of egg ovalbumin in pH 7.4 buffer, supplemented with 365 mg of cholesterol/methyl-beta-cyclodextrin complex, according to the present invention.

The above solutions were heated in thermostated water bath at 42-44° C., followed by heating at 45-47° C., finally at 50-52° C.

It was observed that the control ovalbumin solution precipitated already at 42° C. after several minutes (white coagulated precipitate formed).

The Solution II containing methyl-beta-cyclodextrin showed only slight opalescence at 42° C. after several minutes of treatment. This was expected, as prior arts described the slight stabilizing effect of the methyl-cyclodextrin.

However, to our surprise, it has been found that the same cyclodextrin loaded with cholesterol protected egg ovalbumin significantly better against heat precipitation, the heated solution remained stable clear transparent during a 60 minutes long heating at 42° C.

Then the test solutions No—II. and III. were heat-treated at higher temperature, at 44-47° C. At this temperature in 5 minutes, the methyl-beta-cyclodextrin-containing ovalbumin solution started to aggregate and it showed white precipitation, however, the Solution III. containing cholesterol-loaded methyl-beta-cyclodextrin, according to the present invention, remained stable, transparent solution, even heat treated at 50-52° C. The cholesterol/methyl-beta-cyclodextrin complex supplemented ovalbumin solution was found stable after longer exposure to 50-52° C. heating. A control sample only containing buffer, cholesterol and ovalbumin was not forming a clear solution in the indicated temperature range, since chloesterol did not get dissolved under these circumstances.

The results of the above comparative stability study are listed in Table 4.

TABLE 4
Optical density of pH 7.4 buffered ovalbumin solutions supplemented
with methyl beta-cyclodextrin (MBCD), and with cholesterol
complexed with methyl-beta-cyclodextrin upon heating

optical density of solutions at 405 nm

| T | ovalbumin | ovalbumin | ovalbumin + cholesterol |
|---|---|---|---|
| (° C.) | control | MBCD | complexed with MBCD |
| 25 | 0.011 | 0.008 | 0.010 |
| 42 | 1.70 | 0.160 | 0.011 |
| 45-47 | precipitate* | 1.210 | 0.011 |
| 50-52 | precipitate* | precipitate* | 0.090 |

*not measurable by spectrophotometry

### EXAMPLE 5

### Preparation and Use of Sodium-Palmitate-Loaded Methyl-Beta-Cyclodextrin For Stabilization of Protein Solutions

Sodium-palmitate 3.0 grams and 97 grams of methyl-beta-cyclodextrin with degree of substitution of 12-14 methyl-groups/cyclodextrin ring were dissolved in 95 mL 96 V % ethanol during intense stirring at room temperature. The resulting clear common solution was evaporated to dryness in vacuo at 45° C., using rotatory evaporator, yielding a white amorphous solid. The solid co-evaporate was redissolved in 40 mL of deionized water, ultrasonicated then immediately chilled to minus 65° C. and the water ice was removed by freeze-drying. The product was a white amorphous instantly water-soluble solid, the sodium-palmitate-methyl-beta-cyclodextrin complex. The composition of the solid formulation was 2.9% sodium-palmitate content (determined by HPLC) and a residual water content of 3.2% (determined by Karl-Fischer method).

One gram of the sodium-palmitate-methyl-beta-cyclodextrin complex was dissolved in 2 mL of deionized water by ultrasonication resulting in a transparent, colorless solution. From this stock solution different amounts (5-50 mM) were added to the lysozyme solutions. The control and cyclodextrin complex supplemented solutions protein were investigated during heat treatments at 60° C.

The results of heat stability testing are summarized in Table 5.

TABLE 5
Heat-induced aggregation of lysozyme solutions (by optical density at 405 nm) in the
presence of different amounts of sodium-palmitate/methyl-beta-cyclodextrin complex.

| heat | Ovalbumin aggregation (absorbance at 405 nm) | | | | | | |
|---|---|---|---|---|---|---|---|
| treatment | control | | | 5 mM Na- | 10 mM Na- | 25 mM Na- | 50 mM Na- |
| at 60° C. | (no | 5 mM Na- | MBCD | palmitate in | palmitate in | palmitate in | palmitate in |
| (Hours) | additive) | palmitate | only | complex | complex | complex | complex |
| 0 | 0.017 | 0.022 | 0.022 | 0.018 | 0.021 | 0.020 | 0.021 |
| 0.5 | 0.34 | 0.30 | 0.28 | 0.210 | 0.135 | 0.082 | 0.066 |
| 1.0 | 1.05 | 0.94 | 0.74 | 0.440 | 0.200 | 0.174 | 0.092 |
| 1.5 | 1.58 | 1.34 | 0.89 | 0.780 | 0.310 | 0.190 | 0.100 |
| 2.0 | 1.87 | 1.52 | 1.02 | 0.920 | 0.552 | 0.388 | 0.210 |

### EXAMPLE 6

### Improvement of Thermostability of Proteins by a Cyclodextrin Adenosin-Triphosphate (ATP-Na) Combination

Polyanionic sulfobutylether beta cyclodextrin in a suitable combination with ATP-Na salt improved the thermostability of egg albumin better than the Dexolve™ in phosphate buffered saline.

sulfobutylether-beta-cyclodextrin and ATP alone, respectively. Results are listed in Table 6.

TABLE 6
Change of the optical density of ovalbumin
solutions during storage at 50° C.

| treatment | optical density of egg ovalbumin solutions at 405 nm | | |
|---|---|---|---|
| time | 25% sulfobutylether- | 25% sulfobutylether- | 2.5% |
| (Hrs) | beta-CD | beta-CD + 2.5% ATP | ATP alone |
| 0 | 0.040 | 0.033 | 0.027 |
| 1 | 0.071 | 0.051 | 0.18 |
| 2 | 0.094 | 0.062 | 0.40 |
| 3 | 1.41 | 0.101 | 1.78 |
| 5 | (hazy) | 0.166 | white |
| | | | precipitate |

Application of a combination of a complex forming cyclodextrin and a natural hydrotrope, ATP, was found to result in synergistic stabilization against heat-induced protein aggregation.

### EXAMPLE 7

### Reduction of Agitation-Induced Protein Aggregation by Using Anionic Beta-Cyclodextrin Spermidin Combination

The combination of Dexolve™ sulfobutylether-beta-cyclodextrin (SBECD) with natural polyamine spermidine hydrochloride in 1:1 molar ratio was found to provide synergistic protective effect on growth hormone. Thus 17 g of SBECD and 1.5 g of spermidine were dissolved in 100 mL of phosphate buffered saline solution. Also a separate solution with the same concentration containing only SBECD was made and used in the aggregation test for comparison. Human growth hormone was dissolved in the above buffered solutions and samples were shaken at 800 rpm at room temperature. Samples were withdrawn at different time intervals and their optical density was determined as a function of agitation time. The protective effect of additives against aggregation is summarized in Table 7.

TABLE 7
Optical density of human growth hormone test solutions
during agitation with 800 rpm at room temperature

| | optical density at 450 nm | | | |
|---|---|---|---|---|
| treatment | | 17% | | 1.5% |
| time | no | SBECD + 1.5% | 17% | spermidine |
| (Hrs) | additive | spermidine | SBECD | alone |
| 0 | 0.031 | 0.022 | 0.012 | 0.018 |
| 1 | 0.87 | 0.051 | 0.12 | 0.77 |
| 2 | 1.88 | 0.062 | 0.39 | 1.44 |
| 3 | cloudy | 0.105 | 0.88 | cloudy |
| | solution | | | solution |
| 4 | white | 0.170 | 1.44 (white | white |
| | precipitate | | suspension) | precipitate |

As the above data indicate, SBECD and spermidine alone has also some protective effect against agitation induced protein aggregation, but the combination of a polyamine and the anionic cyclodextrin provides synergistic protective effect.

### EXAMPLE 8

### L-Carnitine/Carboxymethyl-Beta-Cyclodextrin Formulation Preparation and Use For Stabilization of Ovalbumin

4.5 grams of L-carnitine and 96 grams of carboxymethyl-beta-cyclodextrin (CMBCD) were dissolved in 80 mL purified water upon intense agitation at room temperature. The resulting clear solution was lyophilized. The lyophilized L-carnitine carboxymethyl-beta-cyclodextrin formulation had L-carnitine content of 4.4%, and a residual water content of 3.2% (by Karl-Fischer titration)

1 gram of the CMBCD/L-carnitine formulation was re-dissolved in 5 mL of water and added in 15-45 mM amounts to the ovalbumin solution. The heat-induced aggregation of ovalbumin in presence of L-carnitine/CMBCD formulation was followed by optical density measurements during a 60° C. heat treatment of the protein solutions. The results of the heat treatment are shown in Table 8.

TABLE 8
Aggregation of egg ovalbumin solutions in water during a heat treatment at 60°
C. in the presence of L-carnitine/CMBCD formulation (by optical density at 405 nm)

| heat | Ovalbumin aggregation (absorbance at 405 nm) | | | | | | |
|---|---|---|---|---|---|---|---|
| treatment | control | | | 15 mM L- | 20 mM L- | 30 mM L- | 45 mM L- |
| at 60° C. | (no | 15 mM L- | CMBCD | carnitine in | carnitine in | carnitine in | carnitine in |
| (Hours) | additive) | carnitine | only | complex | complex | complex | complex |
| 0 | 0.017 | 0.021 | 0.022 | 0.022 | 0.021 | 0.021 | 0.020 |
| 0.5 | 0.34 | 0.18 | 0.28 | 0.060 | 0.055 | 0.042 | 0.041 |
| 1.0 | 1.05 | 0.88 | 0.74 | 0.083 | 0.065 | 0.056 | 0.032 |
| 1.5 | 1.58 | 1.12 | 0.89 | 0.117 | 0.070 | 0.068 | 0.056 |
| 2.0 | 1.87 | 1.20 | 1.02 | 0.142 | 0.130 | 0.099 | 0.089 |

1. A method for enhancing the physical stability of a protein in an aqueous environment, which comprises adding a cyclodextrin-lipid complex to said aqueous environment in a molar ratio of lipid to cyclodextrin in an amount of 0.01:1 to 1:1 to enhance the physical stability of said protein in said aqueous environment.

2. The method of claim 1, wherein the cyclodextrin in said cyclodextrin-lipid complex comprises one or more cyclodextrins selected from unmodified, alkylated, hydroxyl-alkylated, carboxyalkylated, sulfoalkylated alpha- beta- and gamma-cyclodextrins.

3. The method of claim 1, wherein the lipid in said cyclodextrin-lipid complex comprises one or more lipids selected from sterols, steroids, phospholipids, fatty acids and their naturally occurring derivatives.

4. A method for enhancing the physical stability of a protein in aqueous environment, which comprises adding a complex comprising cyclodextrin and nucleoside phosphate to said aqueous environment in a molar ratio of the nucleoside phosphate to cyclodextrin in an amount of 0.01:1 to 1:1 to enhance the physical stability of said protein in said aqueous environment.

5. The method of claim 4, wherein the cyclodextrin in said complex comprising cyclodextrin and nucleoside phosphate comprises one or more cyclodextrins selected from unmodified, alkylated, hydroxyl-alkylated, carboxyalkylated, sulfoalkylated alpha- beta- and gamma-cyclodextrins.

6. The method of claim 4, wherein the nucleoside phosphate in said complex comprising cyclodextrin and nucleoside phosphate comprises one or more nucleosides selected from natural- and synthetic nucleoside analogue phosphates of different number of phosphate groups.

7. The method of claim 4, wherein said nucleoside is adenosine triphosphate or adenosine diphosphate.

8. A method for enhancing the physical stability of a protein in aqueous environment, which comprises adding a cyclodextrin-amine complex to said aqueous environment in a molar ratio of amine to cyclodextrin in an amount of 0.01:1 to 1:1 to enhance the physical stability of said protein in said aqueous environment.

9. The method of claim 8, wherein the cyclodextrin in said cyclodextrin-amine complex comprises one or more cyclodextrins selected from unmodified, alkylated, hydroxyl-alkylated, carboxyalkylated, sulfoalkylated alpha- beta- and gamma-cyclodextrins.

10. The method of claim 8, wherein the amine in said cyclodextrin-amine complex comprises one or more amines selected from spermidine, spermine, L-carnitine, creatine and taurine.
